(19) United States
(12) Patent Application Publication
(10) Pub. No.: US 2002/0019052 A1
(43) Pub. Date: Feb. 14, 2002

(54) FACS assisted methods for introducing individual chromosomes into cells

(75) Inventors: Edward M. Nolan, San Diego, CA (US); Dietmar P. Rabussay, Solana Beach, CA (US); Gunter A. Hofmann, San Diego, CA (US)

Correspondence Address: Lisa A. Haile, J.D., Ph.D., GRAY CARY WARE & FREIDENRICH LLP, Suite 1600, 4365 Executive Drive, San Diego, CA, 92121-2189, US

(73) Assignee: GENETRONICS, INC.

(21) Appl. No.: 09/974,882

(22) Filed: Oct. 10, 2001

Related U.S. Application Data
(63) Continuation of application No. 09/453,610, filed on Dec. 3, 1999, now abandoned.
(60) Provisional application No. 60/110,950, filed on Dec. 4, 1998.
(60) Provisional application No. 60/110,951, filed on Dec. 4, 1998.

(51) Int. Cl. C12N 15/87; A61K 48/00

(52) U.S. Cl. 435/461; 514/44

(57) ABSTRACT

The present invention provides methods and apparatus for the delivery of at least one chromosome into a cell. Invention methods and apparatus employ FACS or MACS technology for rapidly processing cells and for confirming the introduction of chromosome(s) into a cell. The introduction of the chromosome(s) into the cell is mediated by one or more of a laser, a linear accelerator or electrically induced fusion of a cell and encapsulated chromosome(s). Invention methods provide for the rapid and reliable processing associated with FACS and MACS technology to process thousands of cells a minute, thereby enabling large scale gene transfer.

### RELATED APPLICATIONS

This application claims priority under 35 U.S.C. § 119(e)(1) from provisional U.S. patent application Ser. No. 60/110,951, filed Dec. 4, 1998.

### FIELD OF THE INVENTION

The present invention relates generally to gene transfer into cells and more specifically to the use of electroporation and Flow-Activated Cell Sorting (FACS) to introduce and detect chromosome transfer into cells.

### BACKGROUND OF THE INVENTION

Recent advances in chromosome science have enabled the generation of custom designed mammalian chromosomes to become reality. These “designer” chromosomes can carry any genetic sequence for commercial or clinical relevance and represent a very promising future for genetic manipulation. The introduction of intact single chromosomes (i.e., large protein/DNA complexes) into cells offers unprecedented usefulness as a gene therapy tool, and as a method for generating transgenic animals. Advantages of artificial chromosomes include (1) the introduced chromosome is biologically stable in the cell, thus affording very long, if not permanent, expression; (2) chromosomes will be inherited by daughter cells following cell division; (3) integration of the introduced chromosome into pre-existing chromosomes is not likely and not necessary for stable expression of the delivered gene(s). The avoidance of integration to achieve long term expression eliminates the risk of insertional mutations. However, chromosomes, due to their size, have been very difficult to deliver into cells by any method. Currently, microinjection or microcell fusion is used to delivery chromosomes to cells in vitro but these methods are very laborious and inefficient. Moreover, the yield of cells containing undamaged single chromosomes is low. Accordingly, there exists a need for a method to rapidly and reliably process cells in a manner that provides for the introduction of a single chromosome into a cell in a verifiable manner.

Flow cytometry methods such as fluorescence-activated cell sorting (FACS) are ideal tools to employ in chromosome insertion methods due to their ability to rapidly process and analyze large numbers of individual cells. For example, in traditional flow cytometry, it is common to analyze very large numbers of eukaryotic cells in a short period of time. Newly developed flow cytometers can analyze and sort up to 20,000 cells per second. In a typical flow cytometer, individual particles pass through an illumination zone and appropriate detectors, gated electronically, measure the magnitude of a pulse representing the extent of light scattered. The magnitude of these pulses are sorted electronically into “bins” or “channels”, permitting the display of histograms of the number of cells possessing a certain quantitative property versus the channel number (Davey, H. M., Kell, D. B., Microbiological Reviews, 1996, 60, 4, 641-696). It was recognized early on that the data accruing from flow cytometric measurements could be analyzed (electronically) rapidly enough that electronic cell-sorting procedures could be used to sort cells with desired properties into separate “buckets”, a procedure usually known as fluorescence-activated cell sorting (Davey and Kell, supra).

Fluorescence-activated cell sorting has been primarily used in studies of human and animal cell lines and the control of cell culture processes. Fluorophore labeling of cells and measurement of the fluorescence can give quantitative data about specific target molecules or subcellular components and their distribution in the cell population. Flow cytometry can quantitate virtually any cell-associated property or cell organelle for which there is a fluorescent probe (or natural fluorescence). The parameters which can be measured have previously been of particular interest in animal cell culture.

Flow cytometry has also been used in cloning and selection of variants from existing cell clones. This selection, however, has required stains that diffuse through cells passively, rapidly and irreversibly, with no toxic effects or other influences on metabolic or physiological processes. Since, typically, flow sorting has been used to study animal cell culture performance, the physiological state of cells, and the cell cycle, one goal of cell sorting has been to keep the cells viable during and after sorting. As these methods have been perfected, it has become possible to monitor various cell parameters and sort the cells accordingly, while maintaining viability.

A variety of parameters have been successfully monitored using flow cytometry methods, for example, papers have also been published describing the application of flow cytometry to the detection of native and recombinant enzymatic activities in eukaryotes. Betz et al. studied native (non-recombinant) lipase production by the eukaryote, Rhizopus arrhizus with flow cytometry. (Betz, J. W., Aretz, W., Hartel, W., Cytometry, 1984, 5, 145-150). They found that spore suspensions of the mold were heterogeneous as judged by light-scattering data obtained with excitation at 633 nm, and they sorted clones of the subpopulations into the wells of microtiter plates. After germination and growth, lipase production was automatically assayed (turbidimetrically) in the microtiter plates, and a representative set of the most active were reisolated, cultured, and assayed conventionally (Betz, supra).

Scrienc et al. have reported a flow cytometric method for detecting cloned β-galactosidase activity in the eukaryotic organism, S. cerevisiae. The ability of flow cytometry to make measurements on single cells means that individual cells with high levels of expression (e.g., due to gene amplification via an introduced chromosome) could be detected. In the method reported, a non-fluorescent compound β-naphthol-β-galactopyranoside) is cleaved by β-galactosidase and the liberated naphthol is trapped to form an insoluble fluorescent product. The insolubility of the fluorescent product is of great importance here to prevent its diffusion from the cell. Such diffusion would not only lead to an underestimation of β-galactosidase activity in highly active cells but could also lead to an overestimation of enzyme activity in inactive cells or those with low activity, as they may take up the leaked fluorescent compound, thus reducing the apparent heterogeneity of the population.

One group has described the use of a FACS machine in an assay detecting fusion proteins expressed from a specialized transducing bacteriophage in the prokaryote Bacillus subtilis (Chung, et.al., J. of Bacteriology, Apr. 1994, p. 1977-1984; Chung, et al., Biotechnology and Bioengineering, Vol. 47, pp. 234-242 (1995)). This group monitored the expression of a lacZ gene (encodes β-galactosidase) fused to the sporulation loci in subtilis (spo). The technique used to monitor β-galactosidase expression from spo-lacZ fusions in single cells involved taking samples from a sporulating culture, staining them with a commercially available fluorogenic substrate for β-galactosidase called C8-FDG, and quantitatively analyzing fluorescence in single cells by flow cytometry. In this study, the flow cytometer was used as a detector to screen for the presence of the spo gene during the development of the cells. The device was not used to screen and recover cells having individual chromosomes inserted therein.

Another group has utilized flow cytometry to distinguish between the developmental stages of the delta-proteobacteria Myxococcus xanthus (F. Russo-Marie, et.al., PNAS, Vol. 90, pp.8194-8198, September 1993). As in the previously described study, this study employed the capabilities of the FACS machine to detect and distinguish genotypically identical cells in different development regulatory states. The screening of an enzymatic activity was used in this study as an indirect measure of developmental changes.

The lacZ gene from E. coli is often used as a reporter gene in studies of gene expression regulation, such as those to determine promoter efficiency, the effects of trans-acting factors, and the effects of other regulatory elements in bacterial, yeast, and animal cells. Using a chromogenic substrate, such as ONPG (o-nitrophenyl-(-D-galactopyranoside), one can measure expression of β-galactosidase in cell cultures; but it is not possible to monitor expression in individual cells and to analyze the heterogeneity of expression in cell populations. The use of fluorogenic substrates, however, makes it possible to determine β-galactosidase activity in a large number of individual cells by means of flow cytometry. This type of determination can be more informative with regard to the physiology of the cells, since gene expression can be correlated with the stage in the mitotic cycle or the viability under certain conditions. In 1994, Plovins et al., reported the use of fluorescein-Di-β-D-galactopyranoside (FDG) and C₁₂-FDG as substrates for β-galactosidase detection in animal, bacterial, and yeast cells. (Plovins A., Alvarez A. M., Ibanez M., Molina M., Nombela C., Appl. Environ. Microbiol., 1994, 60, 4638-4641). This study compared the two molecules as substrates for β-galactosidase, and concluded that FDG is a better substrate for β-galactosidase detection by flow cytometry in bacterial cells. The screening performed in this study was for the comparison of the two substrates. The detection capabilities of a FACS machine were employed to perform the study on viable bacterial cells.

Accordingly, it is clear that FACS is a powerful tool that can be employed to determine whether a cell has a given gene or genes (i.e., a chromosome) inserted therein, and to sort the cells while maintaining their viability. Thus, there remains a need in the art for methods to reliably insert a single chromosome into a cell at a rate that enables the rapid processing of large numbers of such insertions. There also remains a need for apparatus that can be used to carry out these methods.

### SUMMARY OF THE INVENTION

In order to address the needs described above, the present invention provides methods and apparatus that employ FACS to process large numbers of cells in a chromosome insertion protocol and to verify the insertion of at least one chromosome into a cell, while maintaining cell viability.

Thus, in one embodiment of the present invention, there are provided methods for the rapid delivery of at least one chromosome into a eukaryotic cell. Invention methods include subjecting a cell to a laser light pulse under conditions sufficient to form a transient hole in the cell plasma membrane, and introducing a single chromosome into the cell through the hole, wherein the cell remains viable after insertion of the chromosome.

In another embodiment of the present invention there is provided an apparatus for the rapid delivery of at least one chromosome into a eukaryotic cell. The apparatus includes a chromosome reservoir, a cell reservoir, a laser light source, an optical tweezer, and a FACS; wherein the cell reservoir is in fluid communication with the FACS via a cell conduit; the cell conduit includes a cell gate for admitting a single cell therethrough; the chromosome reservoir includes a chromosome conduit chromosome reservoir in fluid communication with the optical tweezer, and having a chromosome gate for admitting a single chromosome therethrough; the optical tweezer is in fluid communication with the cell conduit at a point downstream form the cell gate; wherein said laser light source is in optical communication with the cell conduit at a point between the cell gate and the optical tweezer. Thus, an individual cell admitted into the cell conduit via the cell gate, passes by the laser light source for treatment by the laser, then the cell proceeds past the optical tweezer for insertion of the chromosome by the tweezer, and subsequently proceeds to the FACS for confirmation of at least one chromosome insertion into the cell.

Other methods may be employed to insert the chromosome(s) into the cell while employing FACS to process the cells and verify insertion of at least one chromosome. Thus, in another embodiment of the present invention there are provided methods for the rapid introduction of at least one chromosomes into a eukaryotic cell. This embodiment includes contacting at least one chromosome with a single cell, wherein the chromosome has sufficient kinetic energy to cause the chromosome to be introduced into the cell, and wherein the kinetic energy is imparted to the chromosome via a linear accelerator. In another aspect of this embodiment, there are provided methods for the rapid introduction of single chromosomes into eukaryotic cells. This aspect includes passing a single charged chromosome through a linear accelerator under conditions sufficient to accelerate the chromosome through the plasma membrane of a cell, and thereby introducing at least one chromosome into the cell.

In yet another embodiment of the present invention, there are provided methods for the rapid introduction of at least one chromosome into a eukaryotic cell. This embodiment includes contacting an encapsulated single chromosome with a cell, substantially simultaneously with the application of an electric pulse, under conditions sufficient to cause fusion of the encapsulated chromosome with the cell.

### BRIEF DESCRIPTION OF THE FIGURES

FIG. 1 shows a schematic diagram of an example of an apparatus for laser-mediated, FACS-assisted insertion of at least one chromosome into a cell. The exemplary apparatus comprises a chromosome reservoir 1, a chromosome gate 2a for selectively allowing a desired number (preferably one) of chromosomes 5 through the chromosome conduit 2b, to the optical tweezer 9. Cells 7 proceeding from a cell reservoir are admitted through a cell gate 6a to the cell conduit 6b where they are treated by a laser light from a laser 3, that may be aimed by an optional series of one or more deflection mirrors 4. Cells with inserted chromosome(s) 10 then proceed to a FACS (or MACS) unit for analysis.

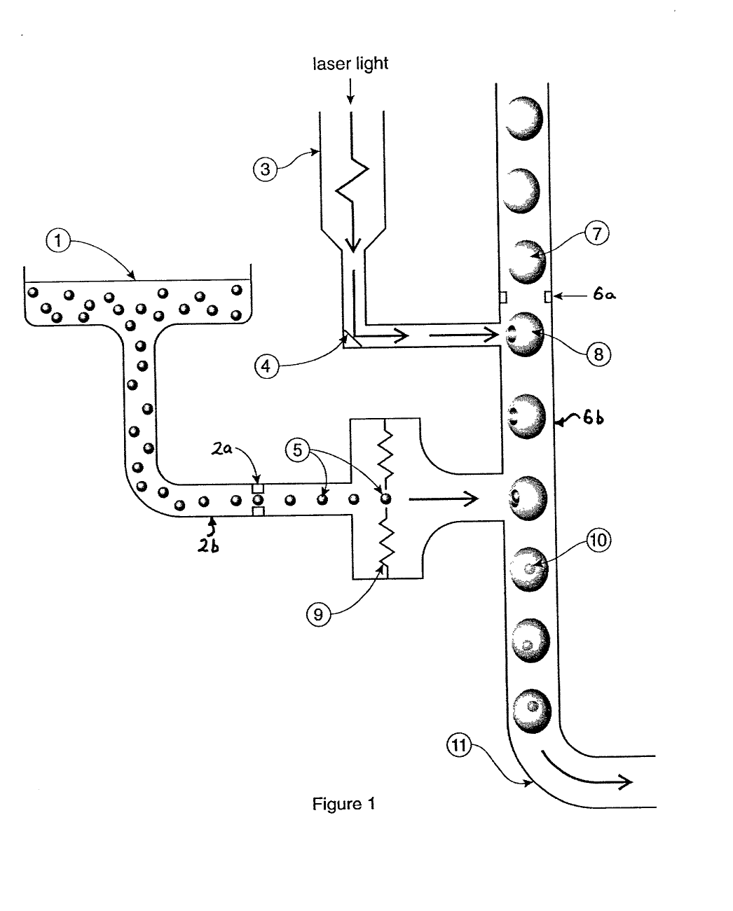

FIG. 2 shows a schematic diagram of an example of an apparatus for linear accelerator-mediated, FACS-assisted insertion of at least one chromosome into a cell. The exemplary apparatus comprises a chromosome reservoir 1, a chromosome gate 2a for selectively allowing a desired number (preferably one) of chromosomes 7 from a chromosome conduit 2b, to linear accelerator 3. Cells 5 proceeding through a cell conduit 4 one at a time from a gated cell reservoir pass by the linear accelerator where they are contacted with accelerated chromosomes (the flow of which may be further mediated by a micropump (step pump)). Cells with inserted chromosome(s) 6 then proceed to a FACS or MACS unit for analysis.

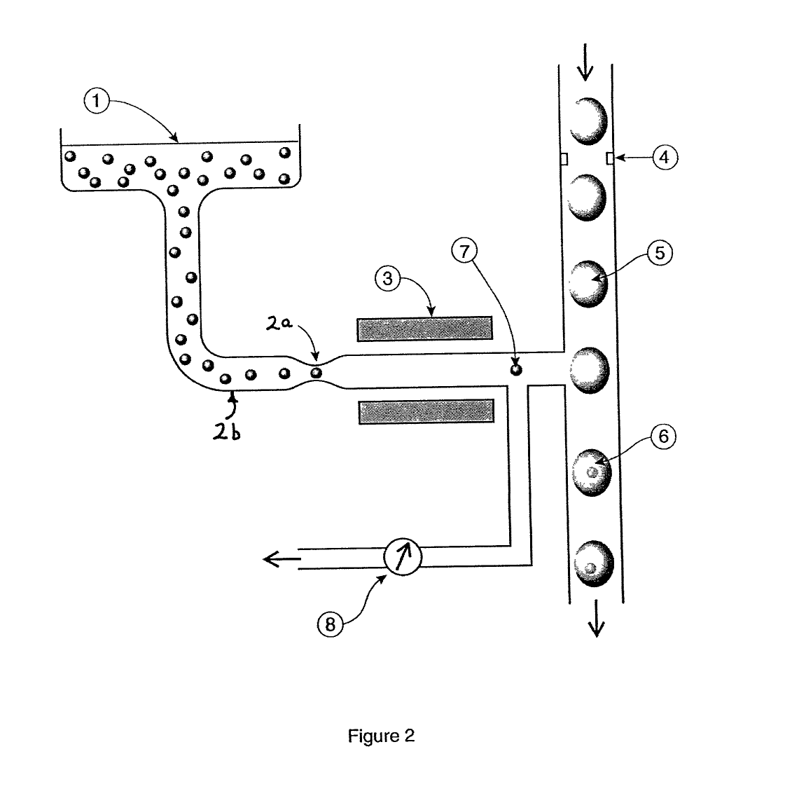

FIG. 3 shows a schematic diagram of an example of an apparatus for cell fusion-mediated insertion of at least one chromosome into a cell. Encapsulated chromosomes 1a from a reservoir 1b proceed through a chromosome conduit 1c to a gate 6 which admits a capsule containing the chromosome(s) into a common conduit 8. Similarly, cells 2a from a cell reservoir 2b proceed through a cell conduit 2c to a cell gate 7 which admits a single cell into the common conduit 8. The encapsulated chromosome(s) and cell proceed substantially simultaneously past an electrode assembly 3 where a charge is applied under conditions sufficient to fuse the capsule and the cell, thereby introducing the chromosome(s) into the cell. The flow of the cells and encapsulated chromosome(s) may be mediated by an optional controlled pump 4. Cells with inserted chromosome(s) then proceed to a reservoir 5 for fused cells (i.e., those cells that have fused with a capsule containing chromosome(s).

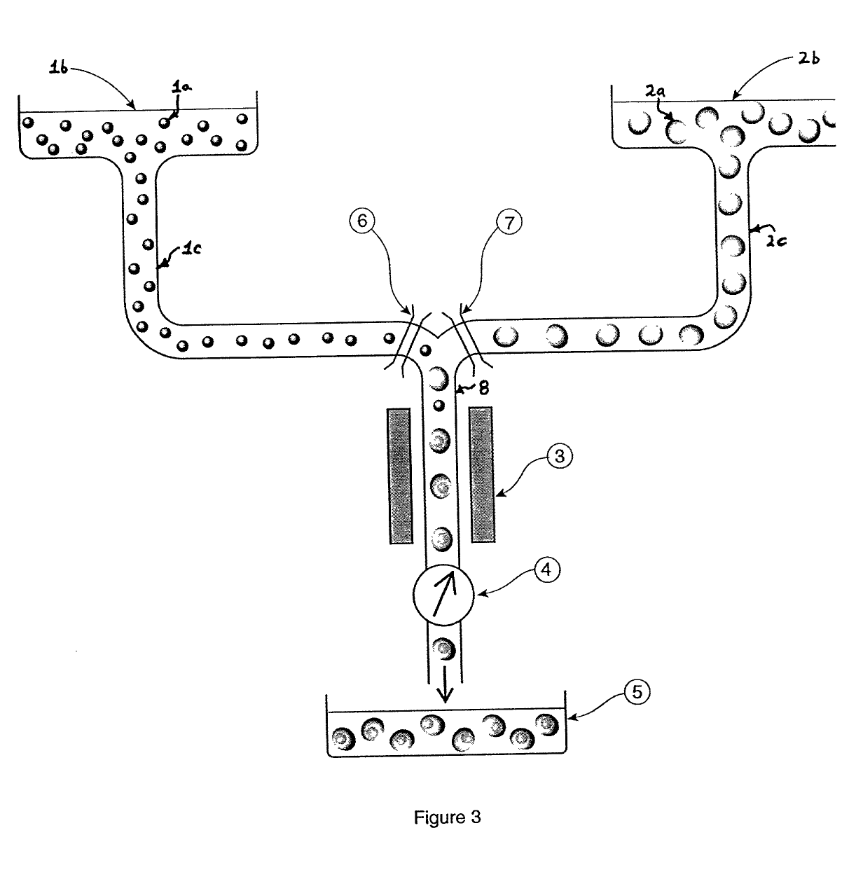

### DETAILED DESCRIPTION OF THE INVENTION

To address the need in the art for methods to introduce at least one chromosome into a cell rapidly and verifiably, in an automated manner that allows large numbers of cells to be processed, the present invention provides novel methods of introducing a single chromosome into a cell, dovetailed with fluorescence-activated cell sorting (FACS) technology for rapid and accurate processing. Magnetic activated cell sorting (MACS) can also be used. The resulting viable cells have introduced into them at least one intact chromosome.

While it may be desirable to insert more than one chromosome into a cell, in preferred embodiments of the present invention, only a single chromosome is inserted into a cell.

This invention differs from fluorescence activated cell sorting, as normally performed, in several aspects. Previously, FACS machines have been employed in the studies focused on the analyses of eukaryotic and prokaryotic cell lines and cell culture processes. FACS has also been utilized to monitor production of foreign proteins in both eukaryotes and prokaryotes to study, for example, differential gene expression, etc. The detection and counting capabilities of the FACS system have been applied in these examples. However, FACS has never previously been employed in a discovery process to screen for and recover eukaryotic cells having single chromosome introduced therein.

As used herein, “chromosome” means a large gene-bearing DNA/protein complex. The size of the chromosome is variable. Technical barriers in the art had previously rendered difficult the insertion of chromosomes comprising greater than about 100 kb in length. In comparison, the typical somatic human chromosome is in the order of 1-10 megabases (i.e., 1,000 to 10,000 kb). Chromosomes also have varying protein content, with the human chromosome being approximately 50% protein by weight. Any chromosomes are suitable for the practice of the present invention, including natural chromosomes, and those modified by human intervention (i.e., artificial chromosomes such as P1-based artificial chromosomes, yeast artificial chromosomes, and the like).

Cells contemplated as recipients or “hosts” for the chromosomal DNA include fibroblasts, parenchyma stem cells both hematopoietic and parenchymal or essentially any cell that can be exploited ex vivo for the purposes of gene delivery, and the like.

In one embodiment of the present invention, there are provided methods for the rapid delivery of single chromosomes into eukaryotic cells. Invention methods include subjecting a cell to a laser light pulse under conditions sufficient to form a transient hole in the cell plasma membrane, and introducing a single chromosome into the cell through the hole, wherein the cell remains viable after insertion of the chromosome.

In one embodiment, the cell to be treated is maintained in a media supplemented with phenol red at a concentration typically found in commercial cell culture media. In this manner, a laser light focussed on the cell membrane excites the phenol red, causing the formation of a transient hole in the cell surface, through which the chromosome is inserted. It is presently preferred that the media further comprise other agents known to those of skill in the art to be required to maintain the chromosomes in their condensed (metaphase) state.

Any laser of suitable energy may be employed in the practice of the present invention. The Argon laser, which typically emits light at 488 nm, serves as an exemplary model. Suitable energies can be generated by lasers having a wavelength in the range of 380 nm up to about 550 nm. It is presently preferred that the laser beam be approximately 488 nm.

Focusing of the laser beam can be carried out by any suitable focussing means. Presently it is preferred that the laser be focussed through an optical lens, such as the 100X objective of a light microscope, or the light can be passed through other optical filters that will reduce and/or focus the light to the needed dimensions.

There are numerous means for determining the successful incorporation of a single chromosome into the cell. It is presently preferred that the verification be made by a FACS machine. Presently, it is preferred that the chromosome be fluorescently labeled. Thus, after insertion of the chromosome, the cell can pass into a recovery chamber where its fluorescent scatter properties are analyzed by the FACS to determine whether one and only one chromosome has been inserted. Current artificial chromosomes are very AT rich due to the fact that they contain a large percentage of pericentric alpha satellite DNA, which is very AT rich. This type of chromosome is identified and sorted by using chromomycin A3 and Hoechst 33258 stains and dual laser high speed flow cytometry. The AT rich chromosomes carry a specific ratio of the dyes and can be identified in this manner.

Another means for utilizing FACS includes assaying for the activity of an enzyme encoded by the introduced chromosome. These methodologies entail the introduction of an appropriate substrate into the host cell. The substrate will depend on the enzyme being assayed. Substrate can be administered to the cells before or during the process of the cell sorting analysis. In either case a solution of the substrate is made up and the cells are contacted therewith. When done prior to the cell sorting analysis, this can be by making a solution which can be administered to the cells while in culture plates or other containers. The concentration ranges for substrate solutions will vary according to the substrate utilized. Commercially available substrates will generally contain instructions on concentration ranges to be utilized for, for instance, cell staining purposes. These ranges may be employed in the determination of an optimal concentration or concentration range to be utilized in the present invention. The substrate solution is maintained in contact with the cells for a period of time and at an appropriate temperature necessary for the substrate to permeate the cell membrane. Again, this will vary with substrate. Instruments which deliver reagents in stream such as by poppet valves which seal openings in the flow path until activated to permit introduction of reagents (e.g. substrate) into the flow path in which the cells are moving through the analyzer can be employed for substrate delivery.

The substrate is one that is able to enter the cell and maintain its presence within the cell for a period sufficient for analysis to occur. It has generally been observed that introduction of the substrate into the cell across the cell membrane occurs without difficulty. It is also preferable that once the substrate is in the cell it not “leak” back out before reacting with the biomolecule being sought to an extent sufficient to product a detectable response. Retention of the substrate in the cell can be enhanced by a variety of techniques. In one, the substrate compound is structurally modified by addition of a hydrophobic tail. In another certain preferred solvents, such as DMSO or glycerol, can be administered to coat the exterior of the cell. Also the substrate can be administered to the cells at reduced temperature which has been observed to retard leakage of the substrate from the cell's interior.

A broad spectrum of substrates can be used which are chosen based on the type of bioactivity sought. In addition where the bioactivity being sought is in the same class as that of other biomolecules for which a number have known substrates, the bioactivity can be examined using a cocktail of the known substrates for the related biomolecules which are already known. For example, substrates are known for approximately 20 commercially available esterases and the combination of these known substrates can provide detectable, if not optimal, signal production. Substrates are also known and available for glycosidases, proteases, phosphatases, and monoxygenases.

The substrate interacts with the target biomolecule so as to produce a detectable response. Such responses can include chromogenic or fluorogenic responses and the like. The detectable species can be one which results from cleavage of the substrate or a secondary molecule which is so affected by the cleavage or other substrate/biomolecule interaction to undergo a detectable change. Innumerable examples of detectable assay formats are known from the diagnostic arts which use immunoassay, chromogenic assay, and labeled probe methodologies.

Other FACS-based detection methods include those based on the presence of a particular mRNA expressed from the introduced chromosome. For example, the inclusion in a chromosome of sequences which result in secondary RNA structures such as hairpins which are designed to flank certain regions of a selected mRNA transcript would serve to enhance the message stability, thus increasing its half life within the cell. Probe molecules may then be employed consisting of oligonucleotides labeled with reporter molecules that only fluoresce upon binding of the probe to a target mRNA molecule. These probes are introduced into the target cells (pre-insertion of the chromosome) using one of several transformation methods. The probe molecules bind to the transcribed target mRNA resulting in DNA/RNA heteroduplex molecules. Binding of the probe to a target will yield a fluorescent signal which is detected and sorted by the FACS machine during the screening process.

It is possible to introduce a single chromosome into a cell utilizing methodologies other than the above-described laser-mediated poration, while still employing FACS technology for rapid processing and verification of single chromosome insertion. One such method is the use of a linear accelerator to impart kinetic energy to charged chromosome. Thus, in another embodiment of the present invention there are provided methods for the rapid introduction of single chromosomes into eukaryotic cells, comprising contacting a single chromosome with a single cell, wherein the chromosome has sufficient kinetic energy to cause the chromosome to be introduced into the cell, and wherein the kinetic energy is imparted to the chromosome via a linear accelerator. This method is referred to herein as “ballistic insertion”. Conditions sufficient to accelerate the chromosome through the plasma membrane of a cell include, magnetic interaction of chromosomes containing metal (iron) atoms or other responsive elements. The acceleration can be induced by, for example, a negatively charged accelerator which thereby operates to repel the magnetically charged and accelerate the chromosome particle toward the target cell.

Following ballistic insertion of the chromosome, FACS is employed to verify insertion of a single chromosome. Thus, as with the laser-mediated methods, the ballistic insertion method can be conducted in an apparatus that comprises a FACS or MACS unit.

Still other methods for insertion of a single chromosome may be employed in the practice of invention methods. Thus, in another embodiment of the present invention, there are provided methods for the rapid introduction of single chromosomes into eukaryotic cells, comprising contacting an encapsulated single chromosome with a cell, substantially simultaneously with the application of an electric pulse, under conditions sufficient to cause fusion of the encapsulated chromosome with the cell.

In addition to the methods described herein, there are also provided apparatus for performing aspects of invention methods. Thus, in one embodiment of the present invention, there are provided apparatus for the rapid delivery of single chromosomes into eukaryotic cells. The apparatus includes a chromosome reservoir, a cell reservoir, a laser light source, an optical tweezer, and a FACS (or MACS); wherein the cell reservoir is in fluid communication with the FACS via a cell conduit; the cell conduit includes a cell gate for admitting a single cell therethrough; the chromosome reservoir includes a chromosome conduit chromosome reservoir in fluid communication with the optical tweezer, and having a chromosome gate for admitting a single chromosome therethrough; the optical tweezer is in fluid communication with the cell conduit at a point downstream form the cell gate; wherein said laser light source is in optical communication with the cell conduit at a point between the cell gate and the optical tweezer. Thus, an individual cell admitted into the cell conduit via the cell gate, passes by the laser light source for treatment by the laser, then the cell proceeds past the optical tweezer for insertion of the chromosome by the tweezer, and subsequently proceeds to the FACS (or MACS) for confirmation of single chromosome insertion into the cell.

In another aspect of the foregoing embodiment, the laser is omitted and a linear accelerator is substituted for the optical tweezer. Thus, in this aspect the apparatus includes a chromosome reservoir, a cell reservoir, a linear accelerator, and a FACS (or MACS); wherein the cell reservoir is in fluid communication with the FACS via a cell conduit; the cell conduit includes a cell gate for admitting a single cell therethrough; the chromosome reservoir includes a chromosome conduit chromosome reservoir in fluid communication with the linear accelerator, and having a chromosome gate for admitting a single chromosome therethrough; the linear accelerator is in fluid communication with the cell conduit at a point downstream form the cell gate. Thus, an individual cell admitted into the cell conduit via the cell gate, proceeds past the linear accelerator for insertion of the chromosome by the linear accelerator, and subsequently proceeds to the FACS (or MACS) for confirmation of single chromosome insertion into the cell.

In yet another aspect of the present invention, there is provided apparatus for practicing the encapsulated chromosome fusion methods described herein. In this aspect, an electroporation apparatus is employed for the permeabilization of target cells. Thus, in this aspect of the invention, the apparatus includes a chromosome reservoir, a cell reservoir, an electroporation apparatus, and a FACS (or MACS); wherein the cell reservoir is in fluid communication with the FACS via a cell conduit; the cell conduit includes a cell gate for admitting a single cell therethrough; an electroporation apparatus is provided in fluid communication with the cell conduit at a point downstream from the cell gate; the chromosome reservoir includes a chromosome gate for admitting a single chromosome therethrough into a chromosome conduit; the chromosome conduit is in fluid communication with the cell conduit at a point substantially the same as electroporation apparatus. Thus, an individual cell admitted into the cell conduit via the cell gate, proceeds past the electroporation apparatus for electropulsing, under conditions sufficient to transiently permeabilize the cell membrane; at this point, the encapsulated chromosome is contacted with the cell under conditions sufficient to cause fusion of the capsule and the cell membrane; and subsequently, the cell proceeds to the FACS (or MACS) for confirmation of single chromosome insertion into the cell

It will be apparent to those skilled in the art that various modifications and variations can be made to the compounds and processes of this invention. Thus, it is intended that the present invention cover such modifications and variations, provided they come within the scope of the appended claims and their equivalents. Accordingly, the invention is limited only by the following claims.

What is claimed is:

1. A method for the delivery of at least one chromosome into a eukaryotic cell, said method comprising subjecting a cell to a laser light pulse under conditions sufficient to form a transient hole in the cell plasma membrane, and introducing at least one chromosome into the cell through said hole, wherein said cell remains viable after introduction of said at least one chromosome.

2. The method according to claim 1, wherein a single chromosome is introduced into said cell.

3. The method according to claim 1, wherein said laser light has a wavelength of about 488 nm.

4. The method according to claim 1, wherein the laser is projected onto the surface of said cell through a microscope lens.

5. The method according to claim 1, wherein said at least one chromosome is greater than about 100 kb in size.

6. The method according to claim 5, wherein said at least one chromosome is in the range of about 100 kb up to about 1,000 kb in size.

7. The method according to claim 5, wherein said at least one chromosome is in the range of about 1,000 kb up to about 30,000 kb in size.

8. The method according to claim 1, wherein said at least one chromosome is less than about 100 kb in size.

9. The method according to claim 1, wherein said at least one chromosome comprises protein.

10. The method according to claim 1, wherein said at least one chromosome comprises in the range of about 40-60% protein by weight.

11. The method according to claim 1, wherein said at least one chromosome comprises about 50% protein by weight.

12. The method according to claim 1, wherein said cells are maintained in a solution comprising phenol red and cell culture material and other agents required to maintain the chromosomes in their condensed (metaphase) state.

13. The method according to claim 12, wherein said laser light is of a wavelength that interacts with phenol red.

14. The method according to claim 1, wherein said insertion of said at least one chromosome is carried out using optical tweezers.

15. The method according to claim 1, wherein said at least one chromosome is fluorescently labeled.

16. The method according to claim 15, wherein insertion of said at least one chromosome is confirmed in a fluorescent activated cell sorter (FACS).

17. An apparatus for the delivery of at least one chromosome into a eukaryotic cell, said apparatus comprising:
a) a chromosome reservoir,
b) a cell reservoir,
c) a laser light source,
d) an optical tweezer, and
e) a FACS;
wherein:
said cell reservoir is in fluid communication with said FACS via a cell conduit, said cell conduit comprising a cell gate for admitting a single cell therethrough,
said chromosome reservoir comprises a chromosome conduit in fluid communication with said optical tweezer, said chromosome conduit having a chromosome gate for admitting at least one chromosome therethrough,
said optical tweezer is in fluid communication with said cell conduit at a point downstream form said cell gate,
wherein said laser light source is in optical communication with said cell conduit at a point between said cell gate and said optical tweezer;
such that an individual cell admitted into said cell conduit via said cell gate, passes by said laser light source for treatment by said laser, then proceeds past said optical tweezer for insertion of at least one chromosome, and subsequently proceeds to said FACS for confirmation of introduction of at least one chromosome into said cell.

18. A method for the introduction of at least one chromosome into a eukaryotic cell, said method comprising contacting at least one chromosome with a single cell, said at least one chromosome having sufficient kinetic energy to cause the chromosome to be introduced into said cell, wherein said kinetic energy is imparted to said at least one chromosome via a linear accelerator.

19. A method for the introduction of at least one chromosome into a eukaryotic cell, said method comprising passing at least one charged chromosome through a linear accelerator under conditions sufficient to accelerate said at least one chromosome through the plasma membrane of a cell, and thereby introducing said at least one chromosome into said cell.

20. An apparatus for the delivery of at least one chromosomes into a eukaryotic cell, said apparatus comprising:
a) a chromosome reservoir,
b) a cell reservoir,
c) a linear accelerator and
d) a FACS;
wherein:
said cell reservoir is in fluid communication with said FACS via a cell conduit, said cell conduit comprising a cell gate for admitting a single cell therethrough,
said chromosome reservoir comprises a chromosome conduit chromosome reservoir in fluid communication with said linear accelerator,
such that an individual cell admitted into said cell conduit via said cell gate, passes by said linear accelerator as at least one chromosome is accelerated therethrough with sufficient kinetic energy to introduce said at least one chromosome into said cell, wherein said cell subsequently proceeds through said FACS for confirmation of introduction of at least one chromosome into said cell.

21. A method for the introduction of at least one chromosome into a eukaryotic cell, said method comprising contacting an encapsulated at least one chromosome with a cell substantially simultaneously with the application of an electric pulse under conditions sufficient to cause fusion of said encapsulated at least one chromosome with said cell.

22. The method according to claim 21, wherein said at least one chromosome is encapsulated in a liposphere, a liposome or a micell.

23. An apparatus for the delivery of at least one chromosome into a eukaryotic cell, said apparatus comprising:
a) a chromosome reservoir,
b) a cell reservoir,
c) an electric field source, a controlled pump, and
d) a collection vessel,
wherein:
said cell reservoir is in fluid communication with a conduit junction via a cell conduit, said cell conduit comprising a cell gate for admitting a single cell therethrough,
said chromosome reservoir is in fluid communication with a conduit junction via a chromosome conduit, said cell conduit comprising a chromosome gate for admitting at least one chromosome therethrough,
such that an individual cell admitted into said cell conduit via said cell gate, and at least one encapsulated chromosome admitted into said chromosome conduit via said chromosome gate, are brought into contact at said junction and proceed through a common conduit past said electric field source where the application of an electric pulse causes the fusing of said encapsulated at least one chromosome and said cell thereby introducing said at least one chromosome into said cell.
